(19) United States
(12) Patent Application Publication
(10) Pub. No.: US 2017/0342458 A1
(43) Pub. Date: Nov. 30, 2017

(54) BIOMASS CONTAINMENT DEVICE

(71) Applicant: UNIVERSITY OF MARYLAND, BALTIMORE COUNTY, BALTIMORE, MD (US)

(72) Inventor: Jeffrey G. GARDNER, Catonsville, MD (US)

(21) Appl. No.: 15/602,815

(22) Filed: May 23, 2017

Related U.S. Application Data
(60) Provisional application No. 62/341,824, filed on May 26, 2016.

(51) Int. Cl. C12Q 1/06; C12M 1/12; C12Q 1/37; C12Q 1/40; C12Q 1/10

(52) U.S. Cl. CPC C12Q 1/06; C12Q 1/40; C12Q 1/10; C12Q 1/37; C12M 25/04; G01N 2333/245; G01N 2333/924; G01N 2333/95

(57) ABSTRACT

A biomass containment device (BCD) and methods of measuring microbial growth or enzyme activity in the presence of insoluble substrates using the BCD is described. The BCD is compatible with microbial growth and enzyme assays, is sterilizable, is reusable, and the size can be varied to fit any container.

### CROSS REFERENCE TO RELATED APPLICATIONS

This application is filed under the provisions of 35 U.S.C. §111(a) and claims priority to U.S. Provisional Patent Application No. 62/341,824 filed on May 26, 2016 in the name of Jeffrey Gardner and entitled “Biomass Containment Device,” which is hereby incorporated by reference herein in its entirety.

### STATEMENT REGARDING FEDERALLY SPONSORED RESEARCH

Portions of this invention may have been made with United States Government support under a grant from the U.S. Department of Energy, Office of Science, Office of Biological and Environmental Research under Award Number DE-SC0014183 and the National Institute of General Medical Sciences Initiative for Maximizing Student Development under Award Number R25-GM55036. As such, the U.S. Government may have certain rights in this invention. Portions of the invention may also have been made with support from the Maryland Technology Development Corporation.

### FIELD OF THE INVENTION

The present invention relates to a customizable biomass containment device (BCD) and methods of measuring microbial growth or enzyme activity in the presence of insoluble substrates using the BCD. The BCD is compatible with microbial growth and enzyme assays, is sterilizable, is reusable, and the size can be varied to fit any container.

### BACKGROUND OF THE INVENTION

The degradation of insoluble polysaccharide-based biomass is ubiquitous in nature and essential for the global cycling of carbon and other nutrients (Field et al. 1998; Leschine 1995). Polysaccharide depolymerization is also an important industrial process for the production of renewable fuels and chemicals (Francesko and Tzanov 2011; Lynd et al. 2002). In both cases, the carbohydrate active enzymes (CAZymes) of ecologically and industrially relevant bacteria are the primary drivers of biomass turnover, particularly that of insoluble (recalcitrant) polysaccharides such as cellulose or chitin (Beier and Bertilsson 2013; Mba Medie et al. 2012). Studying microbial polysaccharide degradation requires the ability to measure microbial growth as these recalcitrant substrates are broken down, however there are challenges making accurate measurements of microbial growth without the insoluble material interfering.

Current methods of physiologically studying recalcitrant polysaccharide degradation are challenging for several reasons including, but not limited to, high background noise due to the insoluble material interspersed with cells, high consumable and reagent cost, insoluble material interference with colorimetric assays, and significant time delay between sampling and data acquisition. Improved measurements in broth-based media are required to advance the study of insoluble polysaccharide degradation, and while many protocols have been used including optical density, protein measurements, and viable cell counts, these methods have disadvantages (Bradford 1976; Sieuwerts et al. 2008; Smith et al. 1985). For example, current optical density readings, while able to track microbial growth in real time, can be erratic upon the mixing of the cells and the insoluble substrate. Protein measurements (e.g., BCA or dye-binding assays) are time-delayed, and require that sample be removed from the growing culture. In addition, reagent compatibility and cost need to be taken into account with protein-based assays. For example, if there is plant or chitinous biomass as the growth substrate, the protein found in these heterogeneous substrates will skew the protein measurements. Colony forming unit (CFU) counting is also a time-delayed measurement, even more so than protein-based measures, and also requires the removal of sample from the growing culture.

Several reports have stated that in vivo studies are required to fully understand insoluble substrate degradation, as solely in vitro studies will not necessarily find the best enzymes or their true biologically relevant functions (Cartmell et al. 2011; Naas et al. 2014; Zhang et al. 2014). For accurate, physiologically relevant analyses to occur, the insoluble substrate must be minimally processed. Further, measuring growth of known and new microbial strains degrading recalcitrant polysaccharides could improve accuracy with a mechanism that segregates the biomass from the growing cells in a way that does not disrupt the experiment or remove sample from the culture. Accordingly, there is currently a need for a customizable substrate and cell separation device, which would provide an option to study microbial growth or enzyme activity using optical density measurements.

### SUMMARY OF THE INVENTION

The present invention relates to customizable biomass containment devices (BCD) that allow interaction between insoluble substrates and microbial cells or enzymes but does not interfere with spectrophotometric measurements.

In one aspect, a biomass containment device (BCD) comprising two parts is described, each part comprising a body having a first end and a second end, wherein the first end is open, and wherein the first end of a first part can be inserted into the first end of a second part resulting in the BCD, wherein the first part and the second part comprise pores for liquid to enter the BCD, and wherein the BCD can accommodate and maintain at least one insoluble substrate such that liquid can enter the BCD through the pores and come in contact with the insoluble substrate contained therein.

In another aspect, a biomass containment device insert (BCDI) comprising a body having a first end and a second end is described, wherein the first end is open, and wherein the BCDI is designed to occupy about half of a microtiter well, wherein the BCDI comprises pores for liquid to enter the BCDI, and wherein the BCDI can accommodate and maintain at least one insoluble substrate such that liquid can enter the BCDI through the pores and come in contact with the insoluble substrate contained therein.

In still another aspect, a method of quantifying microbial growth or enzyme activity on insoluble substrates is described, said method comprising:
inserting an insoluble substrate in a BCD or BCDI as described herein;
inserting the BCD or BCDI into a container, wherein the container comprises a medium comprising a microbial strain or an enzyme;
incubating the medium in the container for an effective time at an effective temperature to induce microbial growth or enzyme activity in the presence of the insoluble substrate;
measuring the microbial growth or the enzyme activity at time intervals using a spectrophotometric technique; and
quantifying the microbial growth or enzyme activity using said measurements.

In yet another embodiment, an assay kit is described, said assay kit comprising a BCD or BCDI as described herein, and lyophilized microbial control strains, optionally pre-packed with an insoluble substrate, wherein the kit can be used to determine if a microbial isolate or optimized strain is able to grow using insoluble substrates as a carbon source.

In another embodiment, an assay kit is described, said assay kit comprising a BCD or BCDI as described herein, and control enzymes, optionally pre-packed with an insoluble substrate, wherein the kit can be used to determine if an enzyme is active in degrading the insoluble substrates as a substrate.

Other aspects, features and embodiments of the invention will be more fully apparent from the ensuing disclosure and appended claims.

### BRIEF DESCRIPTION OF THE FIGURES

FIG. 1A illustrates BCDs used in this study to show that BCDs can be made from several 3-D printable materials, and the Mk 1 design is shown as devices 3-D printed using stainless steel (bottom), nylon (middle), and acrylic (top) in an 18 mm test tube.

FIG. 1B illustrates the Mk 2.1 design BCD, showing separate inner and outer pieces.

FIG. 1C illustrates the Mk 2.2 design BCD, showing a complete and closed BCD.

FIG. 1D illustrates the Mk 2.3 design BCD, showing a large BCD for shake flask experiments.

FIG. 2A illustrates a CAD drawing of the first part of an embodiment of the BCD.

FIG. 2B illustrates the first part (10) and the second part (60) of an embodiment of the BCD before insertion.

FIG. 2C illustrates the first part (10) and the second part (60) of an embodiment of the BCD during insertion.

FIG. 2D illustrates the first part (10) and the second part (60) of an embodiment of the BCD following insertion.

FIG. 3A illustrates a series of CAD drawings for a Biomass Containment Device Insert (BCDI) for a 96-well microtiter plate.

FIG. 3B illustrates an example of the height (left) and the diameter (right) of the BCDI that has been 3-D printed using acrylic plastic.

FIG. 4A illustrates differences in growth analysis measurements of bacterium Cellvibrio japonicus using optical density (OD) measurements (closed circles) and colony forming unit (CFU) measurements (open circles).

FIG. 4B illustrates differences in growth analysis measurements of bacterium Cellvibrio japonicus using OD measurements (closed circles) and protein measurement (open circles).

FIG. 4C illustrates the growth analysis of wild type (closed symbols) and a ΔxylA mutant (open symbols) of bacterium Cellvibrio japonicus in minimal defined medium with glucose as the sole carbon source. Growth experiments were performed with no devices (ND, circles), the Mk 2.1 design BCDs (squares), or the Mk 2.2 design (triangles).

FIG. 4D illustrates the growth analysis of bacterium Cellvibrio japonicus wild type (closed symbols) and a ΔxylA mutant (open symbols) in xylan medium under conditions with no devices (ND, circles), Mk 2.1 BCDs (squares), or Mk 2.2 BCDs (triangles).

FIG. 5A illustrates the comparative growth analyses of bacterium Cellvibrio japonicus using insoluble cellulose without biomass containment, wherein wild type C. japonicus is represented by closed circles, Δcel5BΔcel6A as open squares, and Δgsp as closed inverted triangles. The sole source of carbon used in the growth experiments as filter paper (0.5% w/v) in defined minimal medium.

FIG. 5B illustrates the comparative growth analyses of bacterium Cellvibrio japonicus using insoluble cellulose with biomass containment in the Mk 2.1 device, wherein wild type C. japonicus is represented by closed circles, Δcel5BΔcel6A as open squares, and Δgsp as closed inverted triangles. The sole source of carbon used in the growth experiments as filter paper (0.5% w/v) in defined minimal medium.

FIG. 5C illustrates the comparative growth analyses of bacterium Cellvibrio japonicus using insoluble cellulose with biomass containment in the Mk 2.2, wherein wild type C. japonicus is represented by closed circles, Δcel5BΔcel6A as open squares, and Δgsp as closed inverted triangles. The sole source of carbon used in the growth experiments as filter paper (0.5% w/v) in defined minimal medium.

FIG. 5D represents wild type Cellvibrio japonicus grown in shake flasks using the Mk 2.3 BCDs (closed diamonds) with filter paper as the sole carbon source. Uninoculated flasks (open diamonds) were used to track the background noise generated from insoluble filter paper inside the BCD leaching out.

FIG. 6A illustrates the comparative growth analyses of bacterium Cellvibrio japonicus using the physiologically relevant carbon source corn stover with biomass containment in the Mk 2.1 device, wherein wild type C. japonicus is represented by closed circles, ΔxylA as closed triangles, and Δgsp as closed squares. The sole source of carbon used in the growth experiments was corn stover (0.5% w/v) in defined minimal medium.

FIG. 6B illustrates the comparative growth analyses of bacterium Cellvibrio japonicus using the physiologically relevant carbon source corn stover with biomass containment in the Mk 2.2 device, wherein wild type C. japonicus is represented by closed circles, ΔxylA as closed triangles, and Δgsp as closed squares. The sole source of carbon used in the growth experiments was corn stover (0.5% w/v) in defined minimal medium.

FIG. 6C represents wild type Cellvibrio japonicus grown in shake flasks using the Mk 2.3 BCDs (closed diamonds) with corn stover as the sole carbon source. Uninoculated flasks (open diamonds) were used to track the background noise generated from the insoluble corn stover inside the BCD leaching out.

FIG. 6D characterizes growth of wild type Cellvibrio japonicus (closed symbols) and a ΔxylA mutant (open symbols) either carbon replete or carbon limiting conditions in medium that contains both glucose and xylose. The strains were grown in decreasing amounts of carbon (w/v, 0.25% (circles), 0.1% (squares), or 0.05% (triangles)).

FIG. 7A illustrates a 96-well microtiter plate inoculated with bacterium Esherichia coli bacteria in a nutrient medium. Each well includes a Biomass Containment Device Insert (BCDI).

FIG. 7B illustrates a control experiment, wherein the wells contain the nutrient medium and a BCDI but no microbial cells.

FIG. 7C illustrates the cell growth in the wells of FIG. 7A following 24 hours of incubation.

FIG. 7D illustrates no cell growth in the wells of FIG. 7B following 24 hours of incubation.

FIG. 8 illustrates the low background noise when the BCDIs are present in the microtiter well as well as the nominal variability of using the BCDIs to facilitate microbial growth measurements.

### DETAILED DESCRIPTION, AND PREFERRED EMBODIMENTS THEREOF

The present invention relates to customizable biomass containment devices (BCD) that allow interaction between insoluble substrates and microbial cells or enzymes but do not interfere with spectrophotometric measurements. The BCDs can be manufactured using any known process including, but not limited to, 3-D printing, injection molding, or thermoforming. For the purposes of the instant application, 3-D printing will be discussed but the person skilled in the art will readily understand that other manufacturing processes may be used to manufacture a customizable BCD.

The concept of 3-D printing is nearly 30 years old, but has recently crossed over from being exclusively in the realm of materials science into engineering, biological, and chemical applications (Chia and Wu 2015; Kitson et al. 2012; Symes et al. 2012). The core concept of 3-D printing (also called additive manufacturing in the trade literature) is a digital file of an object modeled in three dimensions that has been sectioned into thin layers (Conner et al. 2015). A 3-D printer reads the digital file and then constructs the object by the deposition or polymerization of a build material, typically plastic or metal. There are currently multiple methods to create 3-D printed objects, with stereolithography (SLA) being a common approach for making microfluidic devices (Ho et al. 2015). Briefly, a controlled beam of ultraviolet light strikes a surface of photosensitive plastic material, e.g., urethane acrylate, and triggers polymerization. A variation of this technique has recently been used to entrap individual microbial cells and create artificial microbial communities (Connell et al. 2013). Both commercial 3-D printing services and 3-D printers themselves are now readily available and increasingly have become a way to rapidly create durable custom components on an individual scale. A robust on-line community for support with the printers and open source sharing of designs, coupled with freely available software, has helped expand the use of 3-D printing to biological research (Baden et al. 2015; Zhang et al. 2013).

As defined herein, “recalcitrant polysaccharides” or “insoluble substrates” or “insoluble biomass” include, but are not limited to lignocellulosic material and chitin. “Lignocellulosic material” is any dry material from a plant and includes, at a minimum, carbohydrates such as cellulose and hemicellulose and/or polyphenolic compounds such as lignin. Lignocellulosic material may also contain xylan, starch, pectin, and the like. Lignocellulosic material includes, but is not limited to: non-woody plant biomass; cultivated crops such as C4 grasses, switch grass, cord grass, rye grass, miscanthus, reed canary grass, or a combination thereof; sugar processing residues such as sugar cane bagasse, beet pulp, or a combination thereof; agricultural residues such as compost, soybean stover, corn stover, rice straw, rice hulls, barley straw, sugar cane straw, corn cobs, wheat straw, canola straw, oat straw, oat hulls, corn fiber, fiber sorghum, animal manure, or a combination thereof; forestry biomass such as recycled wood pulp fiber, sawdust, hardwood, aspen wood, poplar wood, softwood, or a combination thereof. Furthermore, the lignocellulosic feedstock may comprise cellulosic waste material or forestry waste materials such as, but not limited to, newsprint, cardboard and the like. It should be noted that the recalcitrant polysaccharides can be used as is, can be pretreated, and/or can be dewatered and filtered to remove soluble materials. For example, alkali and acid pretreatment liquors can be used to adjust solution pH to a range that will favor microbial growth or enzyme production.

As defined herein, “fits” or “nests” corresponds to inserting a smaller object inside a larger object. In other words, the outside diameter of the smaller object is not as great as the inside diameter of the larger object, such that the smaller object can be inserted into the larger object. Preferably, once inserted, the smaller object does not easily fall out of the larger object.

As defined herein, “nutrients” for supporting microbial growth include, but are not limited to, sugars, minerals, amino acids, nucleotides, vitamins, salts, buffering species, or combinations thereof

As defined herein, “microbe” or “microbial” or “micro-organisms” include a bacteria, yeast (fungi), or archaea.

While not to be construed as limiting, the term “bacteria,” “bacterium” or “bacterial strain” comprises any species in any of the phyla Acidobacteria, Actinobacteria, Aquificae, Armatimonadetes, Bacteroidetes, Caldiserica, Chlamydiae, Chlorobi, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospirae, Planctomycetes, Proteobacteria, Spirochaetes, Synergistetes, Tenericutes, Thermodesulfobacteria, Thermomicrobia, Thermotogae, and Verrucomicrobia, and further encompasses mutants and derivatives of any of the microbial species, such as those produced by known genetic and/or recombinant techniques.

As defined herein, 3-D printing processes include, but are not limited to, (i) the melting or softening of material to produce the layers, e.g., selective laser melting (SLM) or direct metal laser sintering (DMLS), selective laser sintering (SLS), fused deposition modeling (FDM), or fused filament fabrication (FFF), or (ii) the curing of liquid materials using different technologies, e.g., stereolithography (SLA) or digital light processing (DLP). It should be appreciated that although SLA will be discussed herein as a technique of 3-D printing, any of the other known 3-D printing techniques can be used, as readily understood by the person skilled in the art.

The materials that can be printed using a 3-D printing technique include, but are not limited to, thermoplastic materials (e.g., acrylonitrile butadiene styrene (ABS), polycarbonate (PC), polylactic acid (PLA), high density polyethylene (HDPE), polyphenylsulfone (PPSU), poly(meth)acrylate, urethane acrylate, nylon, polyetherimide (PEI), polyether ether ketone (PEEK), high impact polystyrene (HIPS), thermoplastic polyurethane (TPU), polyamides (nylon) and combinations thereof), metals (e.g., aluminum, brass, sterling silver, gold, platinum, titanium, bronze, copper, stainless steel, and combinations thereof), wax, sandstone, ceramics, and combinations thereof

As defined herein, the “container” is intended to correspond to a container that can be inserted into a spectrophotometer, e.g., a cuvette, test tube, a flow-through cell, or microtiter plate, and/or can be a container, e.g., a flask, that the microbial growth or enzyme activity occurs in. It should be appreciated that the microbial growth (or enzyme activity) can occur in a first container, e.g., a flask or a microtiter well, followed by transfer of the liquid to a second container, e.g., a cuvette, for spectrophotometric measurement. Transfer is preferably carried out in a sterile manner based on the nature of the first container and the second container, as readily understood by the person skilled in the art. The containers can be glass or plastic.

As defined herein, the quantification of “enzyme activity” corresponds to the quantification of enzymes produced by micro-organisms or the quantification of enzymes that are not of microbial origin.

Microbial degradation of recalcitrant polysaccharides (e.g., lignocellulose) is currently of great interest for global nutrient cycling studies, as well as biotechnological efforts to cheaply and efficiently produce renewable fuels and chemicals. Previously studied from an almost exclusively biochemical and structural perspective, several reports have stated that in vivo studies are required to fully understand lignocellulose degradation, as solely in vitro studies will not necessarily find the best enzymes or their true biologically relevant functions (Cartmell et al. 2011; Naas et al. 2014; Zhang et al. 2014). Previous attempts to grow saprophytic micro-organisms using insoluble biomass required excessive particle reduction, pre-treatment, and settling times to allow for growth measurements. However for, accurate, physiologically relevant analyses to occur, the substrate must be minimally processed. Measuring growth of microbial strains degrading recalcitrant polysaccharides could improve accuracy with a mechanism to segregate the biomass from the growing cells in a way that does not disrupt the experiment or remove sample from the culture. Understanding the true and accurate degradation of recalcitrant polysaccharides would be useful to many industries including, but not limited to, detergent additive production, bioremediation of toxic materials, renewable fuel, and the pharmaceutical industry.

As discussed below, our data relating to 3-D printed BCDs suggests a cost-competitive solution that makes microbial growth or enzyme activity measurements fast and very reproducible. Additionally, the flexibility of 3-D printing expedites custom device construction for specific experimental conditions. A person skilled in the art will be able to make BCDs having diverse shapes and designs. In addition, there are a growing number of repositories for 3-D printable models and parts for biomedical and biotechnological purposes, including one sponsored by the National Institutes of Health (NIH).

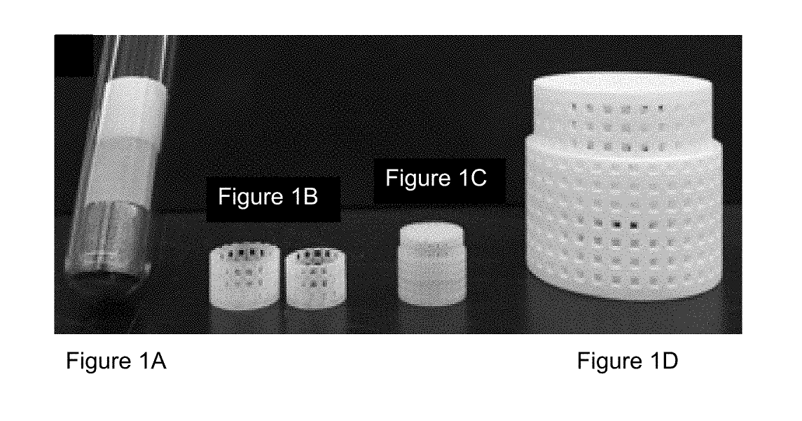

In the first embodiment of the first aspect, a biomass containment device (BCD) is described, said BCD comprising two parts, each part comprising a body having a first end and a second end, wherein the first end in each part is open, and wherein the first end of a first part can be inserted into the first end of a second part (i.e., nesting) resulting in the BCD, wherein the first part and the second part comprise pores for liquid to enter the BCD, and wherein the BCD can accommodate and maintain at least one insoluble substrate such that liquid can enter the BCD through the pores and come in contact with the insoluble substrate contained therein while maintaining the insoluble substrate within the BCD. In a preferred embodiment, the nesting of the first part in the second part results in a BCD wherein upon insertion the first part fits into the second part but can still be pulled out of the second part without excessive force, for example, using fingers, tweezers, pliers or equivalent thereof. Further, upon insertion, the body of the first part preferably extends out of the second part such that it can be grabbed by fingers, tweezers, pliers, or equivalent thereof, as readily understood by the person skilled in the art. The BCD of the first embodiment is illustrated in FIG. 1A-D. FIG. 1B shows the first and the second parts when not nested and FIGS. 1C and 1D show embodiments of the nested first and second parts. It can be seen that the first part and the second part have a cup-like shape, wherein the first end is open and the second end is the base of the cup.

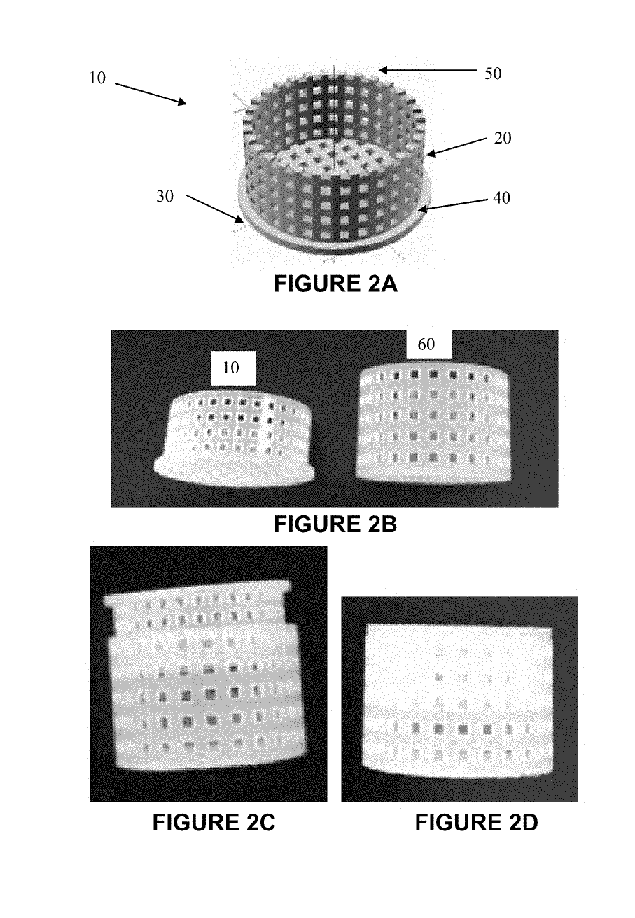

In the second embodiment of the first aspect, the BCD comprises two parts, wherein the first part (10) and the second part (60) each comprise a body, a first end, and a second end, wherein the first end in each part is open, and wherein the first end of the first part (10) can be inserted into the first end of the second part (60) (i.e., nesting) resulting in the BCD, wherein the second end (30) of the first part (10) comprises a rim (40), wherein when the first end (50) of the first part (10) is inserted into the first end of the second part (60), the rim contacts and is substantially flush with the first end of the second part (see, e.g., FIGS. 2A-2D). It can be seen in FIGS. 2A-2D that the first part and the second part have a cup-like shape, wherein the first end is open and the second end is the base of the cup. The second end (30) of the first part (10) has a rim (40), wherein the outside diameter of the body of the second part (60) is the same over the length of the body. Preferably, the body (20) of the first part (10) is shorter in length than the body of the second part (60), allowing the rim (40) of the first part (10) to rest on the first end of the second part (60), wherein the body of the second part and the rim of the first part have substantially the same outside diameter. Advantageously, the rim allows the user to insert and remove the first part from the second part more easily. It should be appreciated by the person skilled in the art that the body of the first part is not required to be shorter in length than the body of the second part such that the rim rests on the first end of the second part. For example, the first part and the second part can nest similar to the first embodiment of the first aspect. Similar to the first embodiment, the first part and the second part of the second embodiment comprise pores for liquid to enter the BCD, wherein the BCD can accommodate and maintain at least one insoluble substrate such that liquid can enter the BCD through the pores and come in contact with the insoluble substrate contained therein while maintaining the insoluble substrate within the BCD.

Although the BCD is described as comprising a first part that nests within a second part, it should be appreciated that a third embodiment of the BCD can comprise a closure of some sort, for example, a screw top, a snap cap, an inner seal cap, a smooth lid, or any other closure known to the person skilled in the art wherein the closure can be opened and closed to insert material into, or remove material from, the BCD. In this embodiment, the BCD would comprise a body and a closure, wherein the body comprises pores for liquid to enter the BCD, and wherein the BCD can accommodate and maintain at least one insoluble substrate such that liquid can enter the BCD through the pores and come in contact with the insoluble substrate contained therein while maintaining the insoluble substrate within the BCD. It should be appreciated that the closure can also comprise pores.

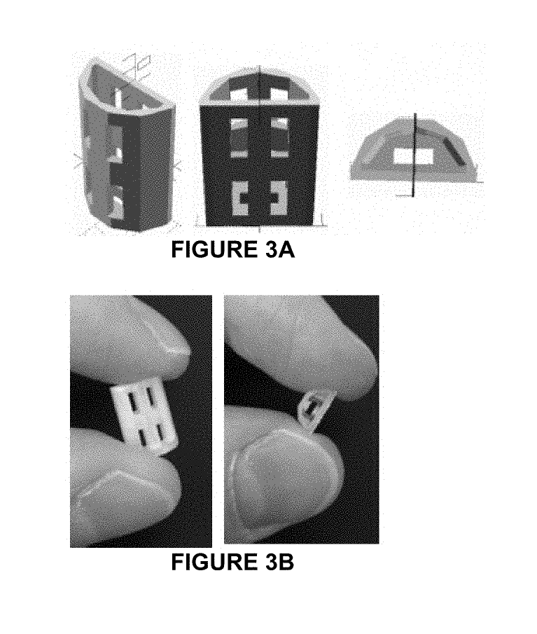

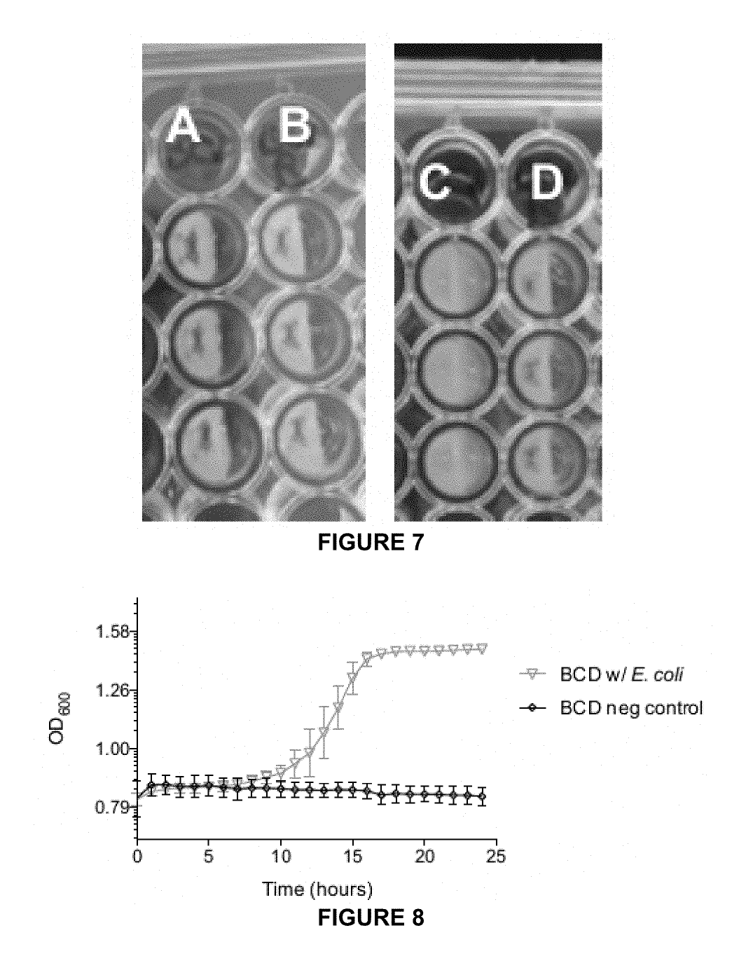

In a fourth embodiment, the BCD fits into a standard 96-well microtiter plate. In this embodiment, the BCD is an insert, or a BCDI, that allows for a high-throughput assay format. A computer-aided drafting (CAD) drawing of the BCD of the fourth embodiment is shown in FIG. 3A, wherein the insert fits into about one half of a well of a microtiter assay plate. It should be noted that the BCDI insert can occupy more than or less than about one half of a microtiter well, but not the whole microtiter well. A portion of the well preferably does not include the BCDI so that liquid can be withdrawn therefrom or a spectrophotometric reading can be obtained, as readily understood by the person skilled in the art. For example, the BCDI can occupy about one half of the length of the well as showing by FIG. 7. As shown in FIG. 3B, the height of the insert can be about 10 mm and the diameter can be about 7 mm, although it should be appreciated that the height and diameter of the BCDI can be adjusted to fit into any size microtiter assay plate. As shown in FIGS. 7A-7D, the BCDIs fit snugly into the wells of the microtiter plate, such that they do not move or rotate if the plate is agitated. BCDIs can comprise only one part because the lid of the microtiter plate will cover the BCDI in the well. That said, a two part BCDI is also contemplated herein, similar to the BCDs described herein.

Regardless of the embodiment, the customizable BCD or BCDI of the first aspect can be printed using a 3-D printing technique or manufactured using another process known in the art. The body of the first and second parts, and hence the BCD, can be circular cylinder, a square cylinder, a polygonal cylinder, or any other shape envisioned by the person skilled in the art for the purpose of containing an insoluble substrate within a container comprising a liquid. The BCDI for a microtiter plate can be a bisected circular cylinder (i.e., a semicircle cylinder), a square cylinder (i.e., a rectangle cylinder), or a polygonal cylinder. The pore shape can be circular, square, rectangular, triangle, and/or polygonal and can be symmetrical or non-symmetrical. The pore size for laboratory bench experiments can be in a range from about 0.1 mm to about 2 mm, preferably about 0.5 mm to about 1.2 mm, even more preferably about 1 mm, as readily determined by the person skilled in the art based on the size of the insoluble substrate. The pore size can be substantially similar throughout the BCD or BCDI or can vary. Further, the pores can be provided on every surface of the parts of the BCD or BCDI, e.g., the body and second end, or can be limited to along just the body of the parts of the BCD or BCDI. It should be appreciated that the BCD or BCDI can be manufactured to be reusable or disposed of after one use. The size of the BCD or BCDI is dependent on the size of the container that contains the liquid (e.g., a spectrophotometric cuvette or test tube), the size of the insoluble substrate, and the ratio of solid to liquid in the experiment, as readily determined by the person skilled in the art. For example, the BCD should be in the container but out of the way of the incident radiation.

In the second aspect, the present invention relates to a method of manufacturing a biomass containment device (BCD) or a BCDI of the first aspect, said method comprising designing a BCD or BCDI of appropriate size and shape; creating a data file based on the design, the data file including a three-dimensional design of the BCD or BCDI; and forming the BCD BCDI from the design file using a rapid manufacturing technique. The rapid manufacturing technique can be one of laser sintering, solid deposition modeling and stereolithography. The design file can be a computer aided design file. The BCD or the BCDI can comprise, consist of, or consist essentially of thermoplastic materials (e.g., acrylonitrile butadiene styrene (ABS), polycarbonate (PC), polylactic acid (PLA), high density polyethylene (HDPE), polyphenylsulfone (PPSU), poly(meth)acrylate, urethane acrylate, polyetherimide (PEI), polyether ether ketone (PEEK), high impact polystyrene (HIPS), thermoplastic polyurethane (TPU), polyamides (nylon) and combinations thereof), metals (e.g., aluminum, brass, sterling silver, gold, platinum, titanium, bronze, copper, stainless steel, and combinations thereof), wax, sandstone, ceramics, and combinations thereof. Alternatively, the BCD or BCDI can be manufactured using other processes known in the art, e.g., injection molding or thermoforming.

In a third aspect, the present invention relates to a method of quantifying microbial growth or enzyme activity on insoluble substrates, said method comprising:
inserting an insoluble substrate in a BCD or BCDI of the first aspect;
inserting the BCD or BCDI into a container, wherein the container comprises a medium comprising a microbial strain or an enzyme;
incubating the medium in the container for an effective time at an effective temperature to induce microbial growth or enzyme activity in the presence of the insoluble substrate;
measuring the microbial growth or the enzyme activity at time intervals using a spectrophotometric technique; and
quantifying the microbial growth or enzyme activity using said measurements.
   Microbial growth or enzyme activity evidences the degradation of insoluble polysaccharide-based biomass. This degradation evidences the potential usefulness of the microbial strain or the enzyme for use in industries including, but not limited to, detergent additive production, bioremediation of toxic materials, renewable fuel, or pharmaceutical industries.

With regards to the third aspect, the method of quantifying microbial growth or enzyme activity can be used to test whether a certain microbial strain will grow or a certain enzyme will be active in the presence of an insoluble substrate. Many factors will be important during the testing process including, but not limited to, the presence of nutrients in the medium, the pH of the medium, the effective temperature of the incubation, the effective time of the incubation, the concentration of the microbial strain or enzymes relative to the amount of insoluble substrate, and any combination thereof. Typically, assay mediums are buffered and the enzymes or microbial strain can be added to the buffer medium. In other words, the test as to whether a certain microbial strain will grow or a certain enzyme will be active in the presence of the insoluble substrate will vary based on any of these factors and adjustments can be used to test for microbial growth or enzyme activity to identify the best process parameters. Other parameters such as time intervals and wavelengths of measurement will be dependent on the specific experiment and equipment and easily determinable by the person skilled in the art. In other words, the technology is useful for enzyme prospecting or microbial strain development in industries including, but not limited to, detergent additive production, bioremediation of toxic materials, renewable fuel, or pharmaceutical industries.

Although the quantification of the microbial growth or enzyme activity on insoluble substrates may be most useful as a batch culture, it should be appreciated by the person skilled in the art that the culture may be continuous as well.

In a fifth aspect, a method of testing if BCDs or BCDIs interfere with microbial growth or enzyme activity is described, said method comprising:
inserting the BCD or BCDI of the first aspect in a first container, wherein the first container comprises a first medium comprising a soluble substrate and a microbial strain or an enzyme;
preparing a second container as a control, wherein the second container comprises a second medium comprising a soluble substrate and a microbial strain or an enzyme, wherein the first medium is the same as the second medium;
incubating the first medium in the first container and the second medium in the second container for an effective time at an effective temperature to induce microbial growth or enzyme activity in the presence of the soluble substrate;
measuring the microbial growth or the enzyme activity at time intervals using a spectrophotometric technique; and
quantifying the microbial growth or enzyme activity using said measurements,
   wherein substantially similar microbial growth or enzyme activity in the first and second containers is evidence that the BCDs or BCDIs are not interfering with microbial growth or enzyme activity.

In a sixth aspect, an assay kit is described, said kit comprising a BCD or a BCDI of the first aspect, optionally pre-packed with an insoluble substrate, and lyophilized microbial control strains. The assay kit can be used to determine if a microbial isolate or optimized strain is able to grow using insoluble substrates as a carbon source.

In a seventh aspect, an assay kit is described, said kit comprising a BCD or a BCDI of the first aspect, optionally pre-packed with an insoluble substrate, and control enzymes. The assay kit can be used to determine if an enzyme is active in degrading the insoluble substrates as a substrate.

In an eighth aspect, a microtiter plate is described, wherein the microtiter plate comprises at least one BCDI of the first aspect positioned in a well.

In a ninth aspect, the BCDs and BCDIs can also be used for studying yeast (fungi) and archaea as well as the enzymes from these micro-organisms. While not to be construed as limiting, the term “fungi,” “yeast,” “yeast strain” or “fungal strain” comprises any species in any of the phyla Blastocladiomycota, Chytridiomycota, Ascomycota, Microsporida, Neocallimastigomycota, Basidiomycota, and Glomeromycota, and further encompasses mutants and derivatives of any of the fungal species, such as those produced by known genetic and/or recombinant techniques. Furthermore, while not to be construed as limiting, the term “archaea” or “archaeal strain” comprises any species in any of the phyla Aenigmarchaeota, Diapherotrites, Nanoarchaeota, Nanohaloarchaeota, Micrarchaeota, Pacearchaeota, Parvarchaeota, Woesearchaeota, Aigarchaeota, Bathyarchaeota, Crenarchaeota, Geoarchaeota, Korarchaeota, Thaumarchaeota, Lokiarchaeota, Thorarchaeota, Odinarchaeota, Heimdallarchaeota, and further encompasses mutants and derivatives of any of the archaeal species, such as those produced by known genetic and/or recombinant techniques.

The present invention relates to the creation and benchmarking of a set of custom biomass containment devices (BCDs) using 3-D printing that greatly facilitate the optical density measurement of microbial growth and enzyme activity during insoluble substrate (i.e., recalcitrant polysaccharide) degradation. In the present set of experiments, it was discovered that a UV-cured acrylic plastic was preferred relative to nylon or stainless steel for printing small pores in cylinder shapes. The acrylic material was also heat tolerant and able to be autoclaved or alcohol sterilized, making the BCDs reusable. The presence of the BCDs in the growth medium did not interfere with microbial growth or diminish the observance of growth phenotypes, which allowed for very reproducible experiments. It was also concluded that 3-D printed BCDs were cost competitive compared to reagents for protein quantitation or colony forming unit (CFU) counting, and had the added benefit of allowing growth measurements to be taken in real time. The use of these devices allows for a standardized, rapid, inexpensive, and reproducible way to use optical density measurements in conjunction with insoluble substrates in the growth medium.

The features and advantages of the invention are more fully illustrated by the following non-limiting examples, wherein all parts and percentages are by weight, unless otherwise expressly stated.

### Example 1

### Design, Manufacture, and Evaluation of Biomass Containment Devices (BCDs)

The stereolithography files (.stl) used in the design of the BCDs were created using a combination of the MakerBot Thingiverse Customizer (http://www.thingiverse.com/apps/customizer) and freely available 3-D computer-aided drafting software (http://www.openSCAD.org). The BCDs were constructed as nested cylinders with an outer diameter of 15 mm and a height of 10 mm. The height dimension was constrained by the height of the light beam coming from the spectrophotometer, which had to pass over the BCD unobstructed through an 18 mm culture tube. The width dimension was constrained by the inner diameter of the 18 mm tube, and a width of 15 mm prevents the device from moving freely once settled to the bottom of the tube (for example, as shown in FIG. 1A). Three designs were evaluated (Mk 1.0, Mk 2.1, Mk 2.2) for 18 mm culture tubes and one design (Mk 2.3) was evaluated for 1 L shake flasks (see, e.g., FIG. 1D). The Mk 1.0 BCDs had only pores at the end of the cylinder and were used for material benchmarking. The Mk 2.1 and Mk 2.3 BCDs had large (1 mm) pores, while the Mk 2.2 BCD had small (0.5 mm) pores on the sides of the device. The Mk 2.x designs were used for growth experiments. Using the 3-D printing service ShapeWays (http://www.shapeways.com), we constructed BCD designs in three materials: nylon (Alumide), acrylic (UV cured), and stainless steel (60% steel, 40% bronze).

### Growth Media and Strains.

Strains of Cellvibrio japonicus (NCIMB #10462, GenBank CP000934.1, previously known as Pseudomonas cellulosa) were grown in defined minimal medium as done previously (Gardner and Keating 2012). Carbon sources were added sterilely at a concentration of 0.5% w/v. Insoluble carbon sources used were Whatman paper (BioRad) or corn stover (Zea mays) obtained from the USDA Sustainable Agriculture Systems Laboratory (Beltsville Md.). Soluble substrates tested were glucose (TekNova) or xylan (Megazyme), prepared according to manufacturer's instructions. Escherichia coli K-12 (CGSC #6300) was grown in lysogeny broth (Bertani 1951; Bertani 2004). All experiments using BCDs were grown in a shaking incubator at 30° C. with a high level of aeration (200 RPM) in either 18 mm glass culture tubes or 1 L glass shake flasks. Growth experiments that did not require biomass containment were performed in a TECAN M200Pro microplate reader.

C. japonicus Mutant Generation.

A C. japonicus ΔxylA in-frame deletion mutant (CJA_RS14735) was generated as previously described (Nelson and Gardner 2015). Confirmation of the correct deletion was performed via PCR screening.

### Optical Density, Colony Forming Unit, and Protein Measurement of Bacteria Growth.

Optical density measurements were obtained at 600 nm with a Spectronic 20D+spectrophotometer (Thermo Fisher). Colony forming unit growth measurements were done as previously described (Gardner and Keating 2010). For total protein assays, cell lysis was performed with the B-PER Bacterial Protein Extraction Reagent (ThermoFisher) and total protein measurements were performed with a 660 nm Protein Assay Kit (Pierce), both used according to manufacturer's instructions.

### Sterilization Procedures.

Biomass containment devices were sterilized by autoclaving for a 30 min steam cycle at 121° C. and 16 psi. An alternative sterilization method used was a combination of bleach and ethanol soaking. Briefly, the biomass containment devices were immersed for 20 minutes in a 10% bleach solution, then rinsed with sterile ddH2O, and then immersed in a 95% ethanol solution for 30 minutes before being placed in sterile glass culture tubes. The tubes were then dried for 10 minutes in a 70° C. oven.

### Biomass Containment Devices (BCDs) Allow for Real-Time Data Acquisition and are a Cost Competitive Choice for Microbial Growth Analyses.

Optical density (OD) readings, colony-forming unit (CFU) counting and protein measurements were compared in terms of temporal delay in data acquisition and cost. The same flasks of C. japonicus were used compare OD and CFU measurements (FIG. 3A). While the quality of data was good for either approach, the OD data was collected in real time while the CFU data could only be collected after 2 days of incubation due to the requirement of viable cell plating and outgrowth. A second independent experiment using protein measurements (bicinchoninic acid, BCA) had a reduced lag time to data acquisition (−15 min) compared to CFU counting, but still was not in real time (see, FIG. 3B). An additional problem with the protein measurement method (BCA) was that it was not sensitive enough to detect early growth (T₀-T₄) (FIG. 3B), which was detectable by both CFU and OD measurements.

We next compared the cost to run these growth analysis experiments. As the tube-based BCDs are small (the interior volume is 1.77 cm³), the cost of manufacture is primarily from the amount of build material used in its construction. One complete BCD (first and second part) ranged in price: $4.76 (nylon; Mk 2.1), $12.24 (acrylic; Mk 2.1), and $15.21 (steel; Mk 2.1). While there are a diversity of protein detection kits that are commercially available that range in cost and complexity, it is universal that they all use consumable reagents. CFU counting also incurs a consumable cost in terms of the plasticware required to perform the dilution and plating series required to enumerate the viable cells in a sample. In contrast the BCDs can be reused, therefore giving substantially more value when used with OD measurements.

### Biomass Containment Devices are Sterilizable and Reusable.

The BCD prototypes (Mk 1.0 design) were evaluated for size relative to the print specifications, ability to be sterilized, and reactivity to microbial cells. All BCD prototypes were printed to the specified size, with less than 0.1 mm variance (data not shown), indicating that the printing process did not add any additional material from design specifications to make them larger. It was noted, however, that some of the 1 mm×1 mm pores in the steel and nylon BCDs were closed off. For these materials the pore size would need to be increased, but this was not seen as advisable as an increased pore size would allow insoluble polysaccharide substrate to escape the BCD. In regards to the outer perimeter of the BCD prototypes, all three material types were able to fit into 18 mm test tubes (see, e.g., FIG. 1A).

We assessed the ability of the BCDs to be sterilized by immersing them in an overnight culture of E. coli K-12 and then sterilized in one of two ways: surface sterilization with bleach and ethanol or autoclaving. After a sterilization procedure, the BCDs were then placed in 5 ml of sterile lysogenic broth (LB) (Bertani 1951) in an 18 mm tube and shaken overnight (30° C., 200 RPM) to evaluate the three sterilization methods. After 24 hr incubation, 100 μL was spread on an LB plate to confirm that no viable cells were present (data not shown). A combination of bleach followed by ethanol immersion was an effective means of sterilization for the acrylic and nylon BCDs only. Advantageously, the steel and the plastic BCDs were able to be autoclave sterilized multiple times with no adverse effects to shape function other than a slight yellowing of the acrylic BCD, which was originally translucent. All three BCD materials were able to sustain multiple overnight (12 hr) incubations in a 70° C. drying oven with no deformation. While evaluating the BCDs for ability to be sterilized we repeatedly observed that the steel material severely discolored and released a brown precipitate into the medium (not shown), therefore we discontinued further analysis of this material. Based on the benchmarking studies, we continued using the acrylic BCDs to assess growth phenotypes of C. japonicus growth using polysaccharide substrates.

Biomass Containment Devices do not Interfere with OD Measurements.

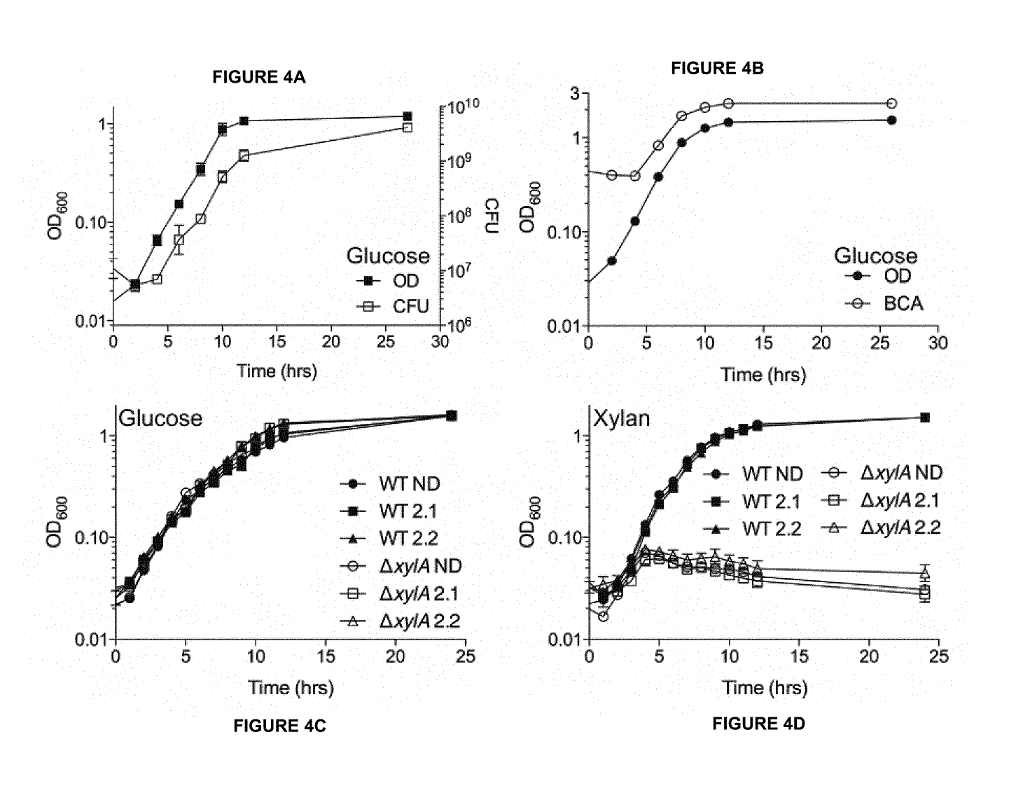

The acrylic BCDs were evaluated for reactivity in a defined minimal medium. If the device material inhibited growth, then it would not be suitable. To assess this, we placed autoclaved acrylic BCDs in 5 ml of defined minimal medium with either glucose or xylan (i.e., a soluble substrate) as the sole carbon source and inoculated the tubes with Cellvibrio japonicus, a recalcitrant polysaccharide degrading bacterium (DeBoy et al. 2008; Gardner et al. 2014). We transitioned to evaluating the Mk 2.1 and Mk 2.2 BCD designs for these experiments. These devices have either small or large pores in the sides and ends, which reduced overall cost compared to the Mk 1.0 BCD and also increase the contact area of the insoluble substrate with the medium. When compared to control cultures that did not have any BCDs present, we found that both the maximum attained OD and growth rate were similar regardless of the presence of the BCDs in the tube when glucose or xylan was used as a carbon source. In order to further test the utility of the BCDs in soluble polysaccharides, we constructed a ΔxylA mutant using our previously described method (Nelson and Gardner 2015). The xylA gene encodes a xylose isomerase and is essential for feeding xylose into the pentose phosphate pathway, therefore a ΔxylA mutant will be unable to use xylan or other xylose-derived oligosaccharides. As shown by FIGS. 4C and 4D, the presence of the BCD does not exacerbate the growth defects of the ΔxylA mutant when grown on either soluble polysaccharides or monosaccharides, although as expected it is unable to grow in xylan containing media. In addition, the BCDs used with wild type C. japonicus did not impact growth under either condition. These data suggest that the acrylic BCDs do not interfere with growth of microbial cells and perform as well or better than conventional cell growth measurements (FIGS. 4A and 4B).

Biomass Containment Devices are Compatible with Insoluble Substrates and Scalable to Vessel Size.

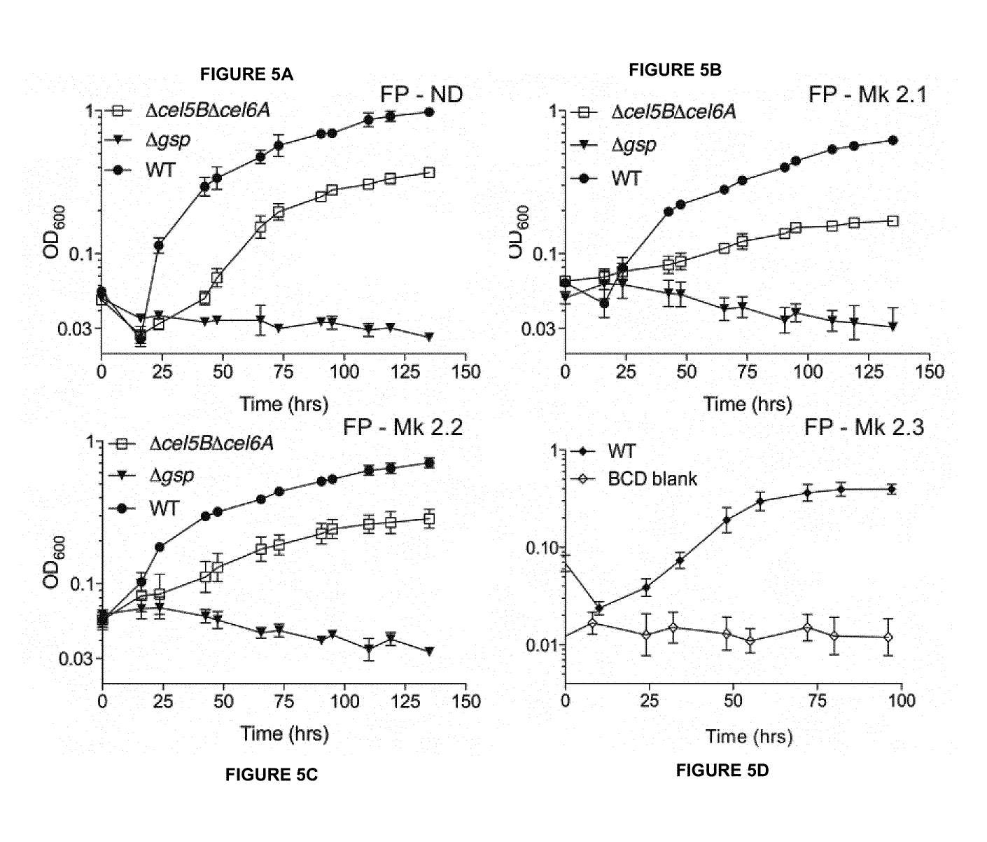

To probe the utility of the BCDs with insoluble polysaccharides, we re-evaluated two mutant strains under biomass containment. A Δcel5BΔcel6A mutant (cel5B is CJA_RS19150; cel6A is CJA_RS19090) and a Δgsp mutant of C. japonicus have moderate and severe growth defects when grown in insoluble cellulose, respectively (Nelson and Gardner 2015), and these strains were tested with cellulose-containing BCDs. The Δgsp mutant has nine genes deleted, gspC (CJA_RS16065), gspD (CJA_RS16060), gspE (CJA_RS16055), gspF (CJA_RS16050), gspG (CJA_RS16045), gspH (CJA_RS18835), gspI (CJA_RS16035), gspJ (CJA_RS16030), and gspK (CJA_RS16025). As expected the Δgsp mutant was unable to grow in cellulose containing medium, as this mutant is deficient for the entire Type II Secretion System, which is an essential component of carbohydrate active enzyme export (Gardner and Keating 2010). The Δcel5BΔcel6A mutant lacks the endoglucanase and cellobiohydrolase enzymes previously shown to be critical for efficient cellulose utilization (Nelson and Gardner 2015). Wild type C. japonicus is unaffected by biomass containment of cellulose and grows well. We chose cellulose as the test case for an insoluble substrate because a previous study examined C. japonicus growth on cellulose without biomass containment (Gardner et al. 2014), which allowed us to conclude that BCDs did not adversely affect degradation and consumption of the insoluble polysaccharide. Our analysis indicates that while the maximum growth for wild type and the Δcel5BΔcel6A mutant was slightly decreased when under biomass containment, the differences in OD were proportional between strains. In addition, we found that there was slightly poorer growth when using the Mk 2.1 (1 mm pores) compared to the Mk 2.2 (0.5 mm pores) designs (FIGS. 5B and 5C, respectively), although both were able to recapitulate the growth phenotypes observed in the control experiment without biomass containment (FIG. 5A). This decrease in observed OD with the Mk 2.1 devices was not due to interference by the BCDs, as both designs had negligible background measurements (not shown).

Large BCDs (Mk 2.3 design) used in 1 L shake flasks for growth experiments were equally effective when scaled up to 500 mL volumes of medium (FIG. 5D). The scalability of the BCDs ensures that large vessels still contain the same percentage of insoluble substrate, typically 0.5% across experiments. While shake flask experiments are not practical for growth analyses (in this case the tube-based BCDs have greater utility), biomass containment in flasks would be useful to facilitate sample removal during an -omics based experiment. Uninoculated flasks containing filter paper without biomass containment started to disperse and create an opaque environment in the flask after 10 hours of shaking (200 RPM), which progressively became more pronounced over the course of the experiment. Conversely, uninoculated BCD control flasks had only minor release of filter paper fragments and like the Mk 2.1 and 2.2 designs, contributed negligibly to background measurements.

### Biomass Containment Devices Facilitate Discovery of Complex Phenotypes During Authentic Lignocellulose Degradation.

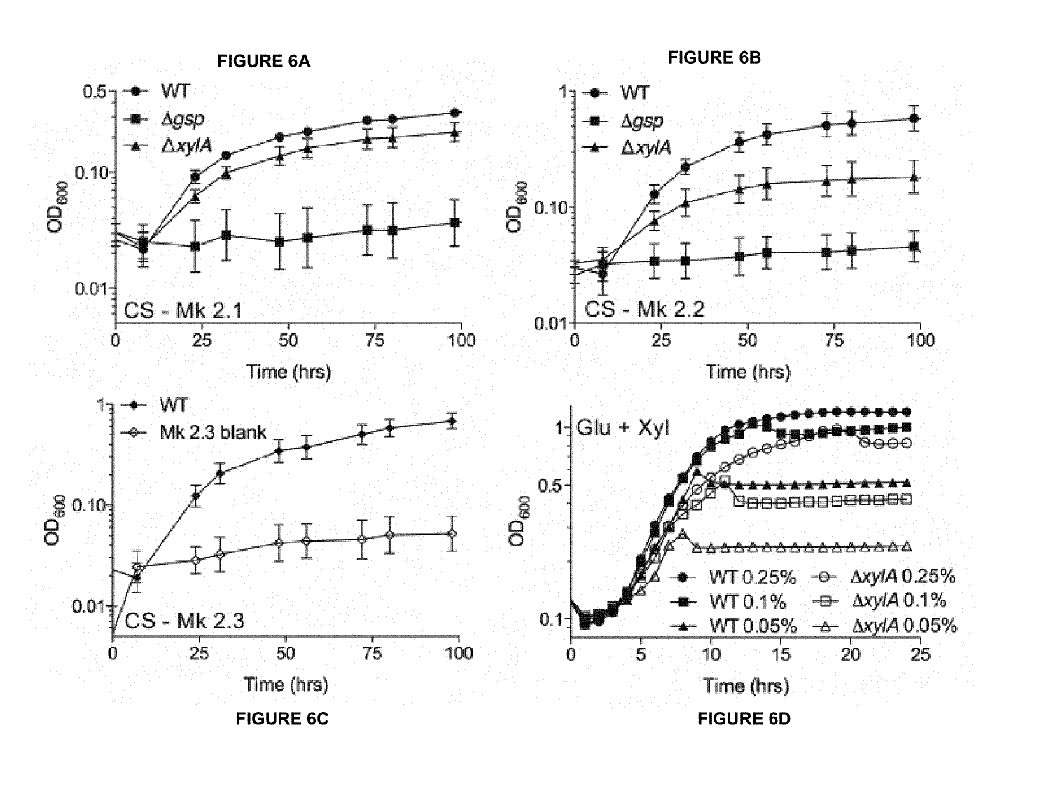

The true utility of the BCDs was evident when testing C. japonicus mutant strains using a physiologically relevant substrate (corn stover). The heterogeneous and insoluble nature of the authentic corn stover substrate previously prevented OD measurements without biomass containment. Shake flask growth experiments with corn stover were greatly facilitated using the Mk 2.3 BCDs. Without biomass containment, particulate material occluded the shake flask by T48 (hours). Some fine particles of corn stover did leach out of the BCD over the course of the experiment, however this background noise contributed negligibly to the overall OD measurements (FIG. 6C).

We evaluated wild type C. japonicus, the newly constructed ΔxylA mutant, and a Δgsp mutant in medium with only authentic corn stover as the sole carbon source under biomass containment. Growth was unable to be measured with no device because of the amount of dispersed insoluble corn stover. The wild type strain of C. japonicus was able to grow well on corn stover, although maximum OD attained in experiments using the Mk 2.1 BCDs was lower than experiments using the Mk 2.2 BCDs, despite the Mk 2.1 design having larger pores. As expected the Δgsp mutant was unable to grow using corn stover, which had been shown previously (Gardner and Keating 2010). Intriguingly, the ΔxylA mutant had a modest, but reproducible growth defect (FIGS. 6A and 6B). The ΔxylA growth defect constituted an approximately 33% reduction in maximum growth using Mk 2.1 BCD, which was determined to be significant by a Student's t-test (p<0.05). The ΔxylA growth defect was more pronounced using Mk 2.2 BCDs (69% reduction in maximum growth from wild type), and also found to be significant. To further probe growth on mixed carbon conditions WT C. japonicus, the ΔxylA mutant, or E. coli K-12 was grown in defined minimal medium with carbon replete conditions (0.25% glucose & 0.25% xylose), moderate carbon limitation (0.1% glucose & 0.1% xylose) or severe carbon limitation (0.05% glucose & 0.05% xylose). The growth analyses indicate that C. japonicus does not have the classical diauxic shift that is present in an E. coli K-12 strain under carbon limitation (FIG. 6D). The E. coli diaxuie phenotype is masked in carbon replete conditions, likely due to exhaustion of another nutrient (NH₃, PO₄, etc.) or accumulation of toxic metabolites before glucose is completely consumed, but manifests under carbon limiting conditions. Furthermore, the growth rate of C. japonicus is faster than E. coli when using either glucose (C. japonicus generation time: 3.4 hrs; E. coli generation time: 4.3 hrs) or xylose (C. japonicus generation time: 2.9 hrs; E. coli generation time: 4.5 hrs) as the sole carbon source. As expected, the ΔxylA mutant grows only 50% as well as WT C. japonicus under carbon limiting conditions, due to the inability of the mutant to utilize xylose. Similar to what was observed using biomass containment with authentic lignocellulose, the ΔxylA mutant has a reproducible growth defect in carbon replete conditions, achieving only 80% of the maximum growth observed for WT C. japonicus (FIG. 6D).

In summary, use of the BCDs allowed authentic biologically relevant substrates to be used with the only manipulation being bending the biomass to fit into the device and sterilization. Containment has the benefit of producing fewer small particles that previously were the source of measurement error. While the Mk 2.3 BCDs move around a great deal more in the flasks than the Mk 2.1 and 2.2 do in the tubes, the background noise for the flask-based experiments was still low. There was virtually no background noise in the tube-based BCD experiments because of the restricted movement of the device at the bottom of the tube. The Mk 2.2 BCDs did perform better with corn stover substrates, as seen by higher overall growth of the C. japonicus strains, which may be a consequence of there being more pores in these devices compared to the Mk 2.1 design because the pore size is smaller.

Further, the ΔxylA mutant had the expected severe growth defect when xylan was the sole carbon source (FIG. 4D), but a striking result obtained was that this mutant also has a growth defect when corn stover was the sole carbon source (FIG. 6B). As the full complement of CAZymes is secreted in the ΔxylA mutant, both soluble hexoses and pentoses are available for uptake. The growth data suggests that xylose consumption is an integral part of the C. japonicus metabolic program during lignocellulose degradation. These data suggests that C. japonicus hexose/pentose metabolism is more similar to P. aeruginosa than E. coli (Rojo 2010). Additional studies of the sugar co-utilization in C. japonicus will be required to further unravel the regulation and mechanisms of substrate preference in this bacterium, but could prove informative because mixed sugar co-utilization has been intensely studied for the production of renewable fuels. Because it affects product yield, it is currently a barrier for complete utilization of saccharified biomass (Zhang et al. 2015).

### Example 2

BCDIs shown in FIGS. 3A-3B were tested in standard 96-well microtiter assay plates using a TECAN M200Pro plate reader. As shown in FIGS. 7A-7D, the BCDIs fit snugly into the wells such that they do not move or rotate if the plate is agitated. FIG. 7A illustrates the wells inoculated with E. coli bacteria in a nutrient medium. FIG. 7B illustrates a control, wherein the wells contain the nutrient medium but no microbial cells. The results following 24 hours of incubation are shown in FIGS. 7C and 7D, wherein the wells comprising the microbial cells are turbid, while the control is not. This verifies that cell growth is not inhibited by the presence of the BCDI in the growth medium.

Referring to FIG. 8, it can be seen that there is very low background noise when the BCDIs are used in the microtiter well (see open diamonds). Further, as noted by the modest standard deviation error bars, the variability of using the BCDIs to facilitate growth measurements is nominal (see open inverted triangles, FIG. 8). The totality of the results suggests that the BCDIs will be valuable for high-throughput screening methods and assays.

### Example 3

The description of the use of the BCDs in enzyme assays hereinbelow uses the BCDIs, however, the tube-based BCDs could be used also.

After the BCDIs have been placed into the wells of a 96-well microtiter assay plate, then an appropriate insoluble substrate will be added to the BCDI. The substrate could be any biological insoluble polymer, such as lignocellulose, chitin, etc. Assay buffer can then be added to each well and finally the enzyme to be tested can be added to the well. After a set amount of incubation at the appropriate temperature, then spectrophotometric readings of the well can be read using a microplate reader for soluble products that have chromogenic or fluorogenic properties. Alternatively, liquid samples can be removed from the well and analyzed by HPLC, TLC, or GC-MS methods. In this manner, the BCDIs can be used to assay the hydrolysis of insoluble substrates and easily remove the soluble products in the sample buffer without contamination from the insoluble material contained in the BCDI. The advantage of using the BCDIs is the high-throughput utility of combining substrate containment of the BCDIs and a large number of simultaneous samples or experiments using the 96-well microtiter plates. Examples of enzymes that could be evaluated with the BCDIs include, but are not limited to, cellulases, xylanases, pectinases, amylases, proteases, chitinases, or any other enzyme that can degrade polymeric substrates. It is possible to evaluate one single enzyme or a combination of several enzymes in each well using the BCDIs.

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

### REFERENCES

Baden T, Chagas A M, Gage G J, Marzullo T C, Prieto-Godino L L, Euler T (2015) Open Labware: 3-D printing your own lab equipment. PLoS Biol 13(3):e1002086 doi:10.1371/journal.pbio.1002086
Beier S, Bertilsson S (2013) Bacterial chitin degradation-mechanisms and ecophysiological strategies. Front Microbiol 4:149 doi:10.3389/fmicb.2013.00149
Bertani G (1951) Studies on lysogenesis. I. The mode of phage liberation by lysogenic Escherichia coli. J Bact 62(3):293-300
Bertani G (2004) Lysogeny at mid-twentieth century: P1, P2, and other experimental systems. J Bact 186(3):595-600
Bradford M M (1976) A rapid and sensitive method for the quantitation of microgram quantities of protein utilizing the principle of protein-dye binding. Anal Biochem 72:248-54
Cartmell A, McKee L S, Pena M J, Larsbrink J, Brumer H, Kaneko S, Ichinose H, Lewis R J, Vikso-Nielsen A, Gilbert H J, Marles-Wright J (2011) The structure and function of an arabinan-specific alpha-1,2-arabinofuranosidase identified from screening the activities of bacterial GH43 glycoside hydrolases. J Biol Chem 286(17):15483-95 doi:10.10741jbc.M110.215962
Chia H N, Wu B M (2015) Recent advances in 3D printing of biomaterials. J Biol Eng 9:4 doi:10.1186/s13036-015-0001-4
Connell J L, Ritschdorff E T, Whiteley M, Shear J B (2013) 3D printing of microscopic bacterial communities. Proc Natl Acad Sci USA 110(46):18380-5 doi:10.1073/pnas.1309729110
Conner B P M G, Martof A N, Rodomsky L M, Rodomsky C M, Jordan D C, Limperos J W (2015) Making sense of 3-D printing: Creating a map of additive manufacturing products and services. Add Manuf 1-4:64-76 doi:http://dx.doi.org/10.1016/j.addma.2014.08.005
DeBoy R T, Mongodin E F, Fouts D E, Tailford L E, Khouri H, Emerson J B, Mohamoud Y, Watkins K, Henrissat B, Gilbert H J, Nelson K E (2008) Insights into plant cell wall degradation from the genome sequence of the soil bacterium Cellvibrio japonicus. J Bact 190(15):5455-63 doi:10.1128/JB.01701-07
Field C B, Behrenfeld M J, Randerson J T, Falkowski P (1998) Primary production of the biosphere: integrating terrestrial and oceanic components. Science 281(5374):237-40
Francesko A, Tzanov T (2011) Chitin, chitosan and derivatives for wound healing and tissue engineering. Adv Biochem Eng/Biotech 125:1-27 doi:10.10007/10_2010_93
Gardner J G, Crouch L, Labourel A, Forsberg Z, Bukhman Y V, Vaaje-Kolstad G, Gilbert H J, Keating D H (2014) Systems biology defines the biological significance of redox-active proteins during cellulose degradation in an aerobic bacterium. Mol Microbiol doi:10.1111/mmi.12821
Gardner J G, Keating D H (2010) Requirement of the type II secretion system for utilization of cellulosic substrates by Cellvibrio japonicus. Appl Enviro Microbiol 76(15):5079-87 doi:10.1128/AEM.00454-10
Gardner J G, Keating D H (2012) Genetic and functional genomic approaches for the study of plant cell wall degradation in Cellvibrio japonicus. Meth Enz 510:331-47 doi:10.1016/13978-0-12-415931-0.00018-5
Gardner J G, Zeitler L A, Wigstrom W J, Engel K C, Keating D H (2012) A high-throughput solid phase screening method for identification of lignocellulose-degrading bacteria from environmental isolates. Biotech Lett 34(1):81-9 doi:10.1007/s10529-011-0742-1
Ho C M, Ng S H, Li K H, Yoon Y J (2015) 3D printed microfluidics for biological applications. Lab on a chip 15(18):3627-37 doi:10.1039/c5Ic00685f
Kitson P J, Rosnes M H, Sans V, Dragone V, Cronin L (2012) Configurable 3D-Printed millifluidic and microfluidic lab on a chip′ reactionware devices. Lab on a chip 12(18):3267-71 doi:10.1039/c21c40761b
Leschine S B (1995) Cellulose degradation in anaerobic environments. Ann Rev Microbiol 49:399-426 doi:10.1146/annurev.mi.49.100195.002151
Lynd L R, Weimer P J, van Zyl W H, Pretorius I S (2002) Microbial cellulose utilization: fundamentals and biotechnology. Microbiol Mol Biol Rev: MMBR 66(3):506-77, table of contents
Mba Medie F, Davies G J, Drancourt M, Henrissat B (2012) Genome analyses highlight the different biological roles of cellulases. Nat Rev Microbiol 10(3):227-34 doi:10.1038/nrmicro2729
Naas A E, Mackenzie A K, Mravec J, Schuckel J, Willats W G, Eijsink V G, Pope P B (2014) Do rumen Bacteroidetes utilize an alternative mechanism for cellulose degradation? mBio 5(4):e01401-14 doi:10.1128/mBio.01401-14
Nelson C E, Gardner J G (2015) In-frame deletions allow functional characterization of complex cellulose degradation phenotypes in Cellvibrio japonicus. Appl Enviro Microbiol doi:10.1128/AEM.00847-15
Rojo F (2010) Carbon catabolite repression in Pseudomonas: optimizing metabolic versatility and interactions with the environment. FEMS Microbiol Rev 34(5):658-84 doi:10.1111/j.1574-6976.2010.00218.x
Sieuwerts S, de Bok F A, Mols E, de vos W M, Vlieg J E (2008) A simple and fast method for determining colony forming units. Lett Appl Microbiol 47(4):275-8 doi:10.1111/j.1472-765X.2008.02417.x
Smith P K, Krohn R I, Hermanson G T, Mallia A K, Gartner F H, Provenzano M D, Fujimoto E K, Goeke N M, Olson B J, Klenk D C (1985) Measurement of protein using bicinchoninic acid. Anal Biochem 150(1):76-85
Symes M D, Kitson P J, Yan J, Richmond C J, Cooper G J, Bowman R W, Vilbrandt T, Cronin L (2012) Integrated 3D-printed reactionware for chemical synthesis and analysis. Nat Chem 4(5):349-54 doi:10.1038/nchem.1313
Zhang C, Anzalone N C, Faria R P, Pearce J M (2013) Open-source 3D-printable optics equipment. PloS ONE 8(3):e59840 doi:10.1371/journal.pone.0059840
Zhang G C, Liu J J, Kong, I I, Kwak S, Jin Y S (2015) Combining C6 and C5 sugar metabolism for enhancing microbial bioconversion. Curr Op Chem Biol 29:49-57 doi:10.1016/j.cbpa.2015.09.008
Zhang X, Rogowski A, Zhao L, Hahn M G, Avci U, Knox J P, Gilbert H J (2014) Understanding how the complex molecular architecture of mannan-degrading hydrolases contributes to plant cell wall degradation. J Biol Chem 289(4):2002-12 doi:10.1074/jbc.M113.527770

What is claimed is:

1. A biomass containment device (BCD) comprising two parts, each part comprising a body having a first end and a second end, wherein the first end is open, and wherein the first end of a first part can be inserted into the first end of a second part resulting in the BCD, wherein the first part and the second part comprise pores for liquid to enter the BCD, and wherein the BCD can accommodate and maintain at least one insoluble substrate such that liquid can enter the BCD through the pores and come in contact with the insoluble substrate contained therein.

2. The BCD of claim 1, wherein the body of the first and second parts can be a circular cylinder, a square cylinder, or a polygonal cylinder.

3. The BCD of claim 1, wherein the pores have a shape selected from the group consisting of circular, square, rectangular, triangle, polygonal, and combinations thereof.

4. The BCD of claim 1, wherein the pores have a size in a range from about 0.1 mm to about 2 mm.

5. The BCD of claim 1, wherein the pores are disposed (i) along the body of each part or (ii) along the body and the second end of each part.

6. The BCD of claim 1, wherein the second end of the first part comprises a rim, wherein when the first end of the first part is inserted into the first end of the second part, the rim contacts and is substantially flush with the first end of the second part.

7. The BCD of claim 1, wherein the BCD comprises material selected from the group consisting of acrylonitrile butadiene styrene (ABS), polycarbonate (PC), polylactic acid (PLA), high density polyethylene (HDPE), polyphenylsulfone (PPSU), poly(meth)acrylate, urethane acrylate, nylon, polyetherimide (PEI), polyether ether ketone (PEEK), high impact polystyrene (HIPS), thermoplastic polyurethane (TPU), polyamides (nylon), aluminum, brass, sterling silver, gold, platinum, titanium, bronze, copper, stainless steel, wax, sandstone, ceramics, and combinations thereof.

7. A biomass containment device insert (BCDI) comprising a body having a first end and a second end, wherein the first end is open, and wherein the BCDI is designed to occupy about half of a microtiter well, wherein the BCDI comprises pores for liquid to enter the BCDI, and wherein the BCDI can accommodate and maintain at least one insoluble substrate such that liquid can enter the BCDI through the pores and come in contact with the insoluble substrate contained therein.

8. The BCDI of claim 7, wherein the body of the BCDI can be a semicircle cylinder, a rectangle cylinder, or about one half of a polygonal cylinder.

9. The BCD of claim 7, wherein the pores have a shape selected from the group consisting of circular, square, rectangular, triangle, polygonal, and combinations thereof.

10. The BCD of claim 7, wherein the pores have a size in a range from about 0.1 mm to about 2 mm.

11. A method of quantifying microbial growth or enzyme activity on insoluble substrates, said method comprising:
inserting an insoluble substrate in a BCD of claim 1;
inserting the BCD into a container, wherein the container comprises a medium comprising a microbial strain or an enzyme;
incubating the medium in the container for an effective time at an effective temperature to induce microbial growth or enzyme activity in the presence of the insoluble substrate;
measuring the microbial growth or the enzyme activity at time intervals using a spectrophotometric technique; and
quantifying the microbial growth or enzyme activity using said measurements.

12. The method of claim 11, wherein the medium comprises an assay buffer.

13. A method of quantifying microbial growth or enzyme activity on insoluble substrates, said method comprising:
inserting an insoluble substrate in a BCDI of claim 7;
inserting the BCDI into a container, wherein the container comprises a medium comprising a microbial strain or an enzyme;
incubating the medium in the container for an effective time at an effective temperature to induce microbial growth or enzyme activity in the presence of the insoluble substrate;
measuring the microbial growth or the enzyme activity at time intervals using a spectrophotometric technique; and
quantifying the microbial growth or enzyme activity using said measurements.

14. The method of claim 13, wherein the medium comprises an assay buffer.

15. An assay kit comprising a BCD of claim 1, and lyophilized microbial control strains, optionally pre-packed with an insoluble substrate, wherein the kit can be used to determine if a microbial isolate or optimized strain is able to grow using insoluble substrates as a carbon source.

16. An assay kit comprising a BCDI of claim 7, and lyophilized microbial control strains, optionally pre-packed with an insoluble substrate, wherein the kit can be used to determine if a microbial isolate or optimized strain is able to grow using insoluble substrates as a carbon source.

17. An assay kit comprising a BCD of claim 1, and control enzymes, optionally pre-packed with an insoluble substrate, wherein the kit can be used to determine if an enzyme is active in degrading the insoluble substrates as a substrate.

18. An assay kit comprising a BCDI of claim 7, and control enzymes, optionally pre-packed with an insoluble substrate, wherein the kit can be used to determine if an enzyme is active in degrading the insoluble substrates as a substrate.
